(19) United States
(12) Patent Application Publication
(10) Pub. No.: US 2013/0045302 A1
(43) Pub. Date: Feb. 21, 2013

(54) Screening Method

(71) Applicant: NOVOZYMES A/S, Bagsvaerd (DK)

(72) Inventors: Christel Thea Jorgensen, Lyngby (DK); Luise Erlandsen, Copenhagen (DK); Lone Dybdal Nilsson, Virum (DK); Kim Borch, Bagsvaerd (DK); Jesper Vind, Vaerlose (DK)

(73) Assignee: NOVOZYMES A/S, Bagsvaerd (DK)

(21) Appl. No.: 13/658,930

(22) Filed: Oct. 24, 2012

Related U.S. Application Data
(63) Continuation of application No. 12/278,004, filed on Sep. 24, 2008, now abandoned.

(30) Foreign Application Priority Data
Feb. 6, 2006 (DK) PA 2006 00158

(51) Int. Cl. A21D 8/04; C12Q 1/44

(52) U.S. Cl. 426/20; 435/19

(57) ABSTRACT

Lipolytic enzymes which improve the properties of dough or baked products generally have a high activity towards lipids which are capable of forming a hexagonal phase, and a screening method was developed on this basis. The improved properties may include a larger loaf volume, an improved shape factor, an improved crumb structure, reduced dough stickiness, improved dough stability and/or improved tolerance towards extended proofing.

### CROSS-REFERENCE TO RELATED APPLICATIONS

This application is a continuation of U.S. application Ser. No. 12/278,004 filed on Sep. 24, 2008 (pending), which is a 35 U.S.C. 371 national application of PCT/EP07/51111 filed Feb. 6, 2007 which claims priority or the benefit under 35 U.S.C. 119 of Danish application no. PA 2006 00158 filed Feb. 6, 2006, the contents of which are fully incorporated herein by reference.

### FIELD OF THE INVENTION

The application relates to a method of screening lipolytic enzymes to identify a candidate for use as a baking additive which can improve the properties of a baked product when added to a dough.

### BACKGROUND OF THE INVENTION

It is known that various properties of dough and baked products can be improved by adding a lipolytic enzyme. A large number of lipolytic enzymes can be obtained from natural sources or by protein engineering, but evaluation in full-scale baking tests is generally quite cumbersome, so screening methods are useful to select candidates for full-scale testing. WO 0032758 discloses a method of screening lipolytic enzymes for use in baking based on their activity towards ester bonds in short-chain and long-chain triglycerides, digalactosyl diglyceride and a phospholipid, particularly phosphatidyl choline (lecithin).

The lipids present in wheat flour are known to consist mainly of triglycerides, phospholipids and galactolipids. The galactolipids are known to consist mainly of mono- and digalactosyl diglyceride (MGDG and DGDG). The phospholipids are known to consist mainly of lyso phosphatidyl choline and phosphatidyl choline, but also include phosphatidyl ethanolamine (PE), N-acyl phosphatidyl ethanolamine (APE) and N-acyl lysophosphatidyl ethanolamine (ALPE).

K. Larsson, pp 237-251, in “lipids in Cereal Technology”, Academic Press, London 1983 indicates that MGDG forms a reverse hexagonal phase while DGDG forms a lamellar phase, and that there is a transition from lamellar to reverse hexagonal phase above a certain a critical weight ratio of MGDG:DGDG, and that the lamellar one is crucial for good baking properties.

“Interactions: The Key to Cereal Quality”, Homer & Hoseney (1998) at page 135 indicates that among the wheat lipids, some favor a lamellar phase and others a reverse or H_{II} type hexagonal phase.

### SUMMARY OF THE INVENTION

The inventors have found that lipolytic enzymes which improve the properties of dough or baked products generally have a high activity towards lipids which are capable of forming a hexagonal phase, and they have developed a screening method on this basis. The improved properties may include a larger loaf volume, an improved shape factor, an improved crumb structure, reduced dough stickiness, improved dough stability and/or improved tolerance towards extended proofing.

Accordingly, the invention provides a method of selecting a lipolytic enzyme for use as an additive to dough, comprising:

a) contacting at least one lipolytic enzyme with a first lipid which is capable of forming a hexagonal phase and with a second lipid which is incapable of forming a hexagonal phase,

b) detecting hydrolysis of ester bonds in each lipid, and

c) comparing the activity towards the first lipid and the second lipid, and

d) selecting a lipolytic enzyme having a higher hydrolytic activity towards the first lipid than the second lipid,

with the proviso that the first lipid is not APE when the second lipid is PC.

The invention also provides a method of selecting a lipolytic enzyme for use as a baking additive, comprising:

a) incubating at least one lipolytic enzyme with a first lipid as defined above,

b) detecting hydrolysis of an ester bond in the lipid, and

c) selecting a lipolytic enzyme which can hydrolyze at least 90% of the lipid.

The invention also provides a method of preparing a dough by adding the selected enzyme, and a method and preparing of baking the dough to prepare a baked product.

### DETAILED DESCRIPTION OF THE INVENTION

### Screening System

In the screening method of the invention, lipolytic enzymes are tested by incubating them with a first lipid and a second lipid and detecting hydrolysis of ester bonds in the two lipids after the incubation. The hydrolytic activities towards the two lipids are compared, and a lipolytic enzyme is selected which has a high activity towards the first lipid compared to the second lipid, e.g. a higher activity towards the first lipid than the second lipid.

The lipolytic enzymes may be incubated with each lipid in purified form. The reaction may be carried out for 30 minutes at 25° C. at a substrate concentration of 0.5-1.5 mM and a concentration of the lipolytic enzyme corresponding to an optical density at 280 nm of 0.4, 0.04 or 0.004, particularly 0.04. The hydrolysis of an ester bond may be determined, e.g., as disclosed in Danish patent application WO 2005/040410. The incubation and determination may also be done with each lipid in a plate assay, e.g. as described later in this specification.

The lipolytic enzymes may also be incubated with lipid in a dough or in a polar lipid fraction, e.g. as described in the HPLC method or an example below. The selected enzyme may be one that hydrolyzes at least 90% (particularly at least 95%) of the first lipid after 45-70 minutes at 32° C. at a dosage of 0.1-5 mg enzyme protein per kg flour, particularly 0.17-0.5 mg/kg.

### First Lipid

The first lipid is monogalactosyl diglyceride (MGDG), N-acyl phosphatidyl ethanolamine comprising an unsaturated acyl (APE), phosphatidyl ethanolamine comprising an unsaturated acyl (PE), or phosphatidic acid. The method of the invention detects hydrolysis to form monogalactosyl monoglyceride (MGMG), N-acyl lysophosphatidyl ethanolamine (ALPE), lysophosphatidyl ethanolamine (LPE) or lyso-phosphatidic acid. Thus, the screening method of the invention selects lipolytic enzymes with a relatively high activity towards a lipid which is capable of forming a reverse or H_{II} type hexagonal phase.

The first lipid may comprise an unsaturated acyl, particularly polyunsaturated, which is preferably straight-chain with 16-20 carbon atoms, such as oleoyl (C18:1), linoleoyl (C18:2) or linolenoyl (C18:3).

### Second Lipid

The second lipid is digalactosyl diglyceride (DGDG), phosphatidyl choline (PC), N-acyl lysophosphatidyl ethanolamine (ALPE), phosphatidyl myoinositol (PI), phosphatidyl serine (PS) or a triglyceride. Further the second lipid may be phosphatidyl ethanolamine not comprising an unsaturated acyl, N-acyl phosphatidyl ethanolamine not comprising an unsaturated acyl, or phosphatidyl glycerol (PG). Thus, the screening method of the invention selects lipolytic enzymes with a relatively low activity towards a lipid which is capable of forming a lamellar phase. In a preferred embodiment the lipolytic has a relatively low activity towards diacetyl tartaric acid esters of monoglycerides and/or towards sodium stearoyl lactylate.

### Use of Screening Results

A lipolytic enzyme may be selected according to the invention and may be used by adding it to a dough and baking the dough to make a baked product. The enzyme may be added at a dosage of 0.05-50 mg enzyme protein per kg of flour, such as 0.05-25 mg enzyme protein per kg of flour, preferably 0.05-10 mg enzyme protein per kg of flour, particularly 0.1-0.5 mg/kg. This may be evaluated by determining properties such as loaf volume, shape factor, crumb structure and/or dough stability e.g. tolerance towards extended proofing by conventional methods, e.g. as described in WO 0032758.

Optionally, an additional enzyme may also be added to the dough. The additional enzyme may be another lipolytic enzyme, an amylase, an amyloglucosidase, a cyclodextrin glucanotransferase, or the additional enzyme may be a peptidase, in particular an exopeptidase, a transglutaminase, a cellulase, a hemicellulase, in particular a pentosanase such as xylanase, a protease, a protein disulfide isomerase, a glycosyltransferase, a branching enzyme (1,4-alpha-glucan branching enzyme), a 4-alpha-glucanotransferase (dextrin glycosyltransferase), a lactase (galactosidase), or an oxidoreductase, e.g., a peroxidase, a laccase, a glucose oxidase, a pyranose oxidase, a lipoxygenase, an L-amino acid oxidase or a carbohydrate oxidase. The amylase may be a fungal or bacterial alpha-amylase, e.g. from Bacillus, particularly B. licheniformis or B. amyloliquefaciens, or from Aspergillus, particularly A. oryzae, a beta-amylase, e.g. from plant (e.g. soy bean) or from microbial sources (e.g. Bacillus). The amylase may be an anti-staling amylase, as described in WO 9953769, i.e. an amylase that is effective in retarding the staling (crumb firming) of baked products, particularly a maltogenic alpha-amylase, e.g. an amylase as described in WO 9104669 or U.S. Pat. No. 6,162,628.

### Dough

The dough generally comprises wheat meal or wheat flour and/or other types of meal, flour or starch such as corn flour, corn starch, rye meal, rye flour, oat flour, oat meal, soy flour, sorghum meal, sorghum flour, potato meal, potato flour or potato starch.

The dough may be fresh, frozen or par-baked.

The dough is typically leavened, e.g. by use of chemical leavening agent (such as sodium bicarbonate) or a yeast culture such as Saccharomyces cerevisiae (baker's yeast).

The dough may also comprise other conventional dough ingredients, e.g.: proteins, such as milk powder, gluten, and soy; eggs (either whole eggs, egg yolks or egg whites); an oxidant such as ascorbic acid, potassium bromate, potassium iodate, azodicarbonamide (ADA) or ammonium persulfate; an amino acid such as L-cysteine; a sugar; a salt such as sodium chloride, calcium acetate, sodium sulfate or calcium sulfate.

The dough may comprise fat (triglyceride) such as granulated fat, oil, butter fat or shortening, but the invention is particularly applicable to a dough where less than 1% by weight of fat (triglyceride) is added, and particularly to a dough which is made without addition of fat.

The dough may further comprise an emulsifier such as mono- or diglycerides, diacetyl tartaric acid esters of mono- or diglycerides, sugar esters of fatty acids, polyglycerol esters of fatty acids, lactic acid esters of monoglycerides, acetic acid esters of monoglycerides, polyoxyethylene stearates, or lysolecithin, but the invention is particularly applicable to a dough which is made without addition of emulsifiers (other than optionally phospholipid).

### METHODS

### Plate Assay

### Preparation of Lecithin Plates pH 5.5

10 g agar in 0.1 M tri-sodium citrate dihydrate buffer (pH 5.5) in a total of 1 liter was heated in microwave oven until agar was dissolved. Then 6 g lecithin (L-a-phosphatidyl choline 95%) and 2 ml 2% crystal violet was added. The mixture was treated with an ultrathorax until lecithin was dispersed, where after it was poured onto lids for microtiter-plates.

### Preparation of APE/ALPE Plates pH 5.5

1 g agarose was added in 50 m1 H₂O and heated in water bath at 65° C. until agarose was dissolved.

0.5 g APE/ALPE (galactolipids extracted from wheat flour) was added to a 0.2 M tri-sodium citrate dihydrate buffer (pH 5.5) and heated in water bath at 65° C. 0.1 ml 2% crystal violet was added and triton-x-100 was added to a concentration of 0.1%. The two solutions were mixed and the mixture was treated with an ultrathorax until APE/ALPE was dispersed, where after it was poured onto lids for microtiter-plates.

### Preparation of monogalactosyl diglyceride (MGDG) Plates pH 5.5

1 g agarose was added in 50 m1 H₂O and heated in water bath at 65° C. until agarose was dissolved.

0.5 g MGDG was added to a 0.2 M tri-sodium citrate dihydrate buffer (pH 5.5) and heated in water bath at 65° C. 0.1 ml 2% crystal violet was added. The two solutions were mixed and the mixture was treated with an ultrathorax until MGDG was dispersed, where after it was poured onto lids for microtiter-plates

### Preparation of Digalactosyl diglyceride (DGDG) Plates pH 5.5

1 g agarose was added in 50 m1 H₂O and heated in water bath at 65° C. until agarose was dissolved.

0.5 g DGDG was added to a 0.2 M tri-sodium citrate dihydrate buffer (pH 5.5) and heated in water bath at 65° C. 0.1 ml 2% crystal violet was added. The two solutions were mixed and the mixture was treated with an ultrathorax until DGDG was dispersed, where after it was poured onto lids for microtiter-plates

### Preparation of phosphatidyl ethanolamine (PE) Plates pH 5.5

1 g agarose was added in 50 m1 H₂O and heated in water bath at 65° C. until agarose was dissolved.

0.5 g PE was added to a 0.2 M tri-sodium citrate dihydrate buffer (pH 5.5) and heated in water bath at 65° C. 0.1 ml 2% crystal violet was added. The two solutions were mixed and the mixture was treated with an ultrathorax until PE was dispersed, where after it was poured onto lids for microtiter-plates

### Screening of Lipolytic Enzymes

Aspergillus transformants expressing different lipolytic variants were inoculated in 0.2 ml YPM growth media in microtiter plates and grown for 3 days at 34° C.

96 holes were created in the PE plates, MGDG plates, DGDG plates, Lecithin plates and the APE/ALPE plates. 5 micro-I of culture supernatant was transferred to a hole on each 15 plate and incubated at 37° C. for 20 hours. The results were expressed semi-quantitatively by to size of the clearing zone.

Those lipolytic variants having activity preferable on the lipids APE/ALPE, PE and/or MGDG as compared to the lipids lecithin and DGDG were selected for further baking tests.

### HPLC Assay

Flour lipids are extracted with an excess of MeOH and subsequently fractionated on a column packed with silica gel (Merck, Silica gel 60, 4×30 cm. The non-polar lipids are removed by hexane followed by ethyl acetate, and the polar lipid fraction is afterwards isolated by running MeOH through the column.

The polar lipid fraction is used as substrate in the HPLC assay. Approximately 0.2-0.5 g polar lipid mix (and possibly additional Lecithin) is emulsified in 10 ml NaOAc buffer pH 5. 50 micro-I enzyme solution is incubated with 500 micro-I substrate solution for 30-180 minutes at 30° C. After incubation the enzyme/substrate mixture is inactivated by heating to 95° C. for 5 minutes. 100 micro-I of the inactivated sample is dissolved in 900 micro-I CHCl₃/MeOH (1:1). The solution is centrifuge and analyzed by HPLC (Varian 250×6.4 mm×¼, Microsorb-MV 100 Å-5 micro-m Si, Analytical Instruments). Mobile phases: A: 80% CHCl₃, 19.5% MeOH, 0.5% NH₄OH, B: 60% CH₃Cl, 33.5% MeOH, 0.5% NH₄OH, 5.5% H₂O, running with gradient. Detector: Sedere, Sedex 75 light scattering, Temp 40° C., pressure 3.5 bar.

### EXAMPLES

### Example 1

Five variants were prepared by amino acid modification and were tested in baking and lipoid hydrolysis. In the baking tests, the loaf volume was evaluated on a scale from A (good volume improving effect) to E (almost no volume improving effect).

Lipid hydrolysis was tested in a plate assay with APE/ALPE as described above and by the method disclosed in Danish patent application WO 2005/040410 for 30 minutes at 25° C. with MGDG and APE as substrates at 1.5 mM using lipolytic enzyme A280 =0.04. Results are given as 0 or on a scale from * (very low activity) to ***** (very high activity).

| | Plate | Method of WO 2005/040410 | | Baking |
|---|---|---|---|---|
| | APE/ALPE | MGDG | APE | Volume |
| Variant 1 | 0 | * | 0 | E |
| Variant 2 | 0 | * | * | D |
| Variant 3 | ***** | **** | ***** | A |
| Variant 4 | ***** | ***** | **** | A |
| Variant 5 | ***** | **** | ***** | A |

The results show that a high activity towards MGDG and APE correlates with good baking performance.

### Example 2

### Lipolytic Enzyme Samples

Ten lipolytic enzymes were tested. They included two monocomponent enzymes isolated from natural sources and eight variants obtained by amino acid modification of these two.

### Dough Preparation

Doughs were prepared according to the European straight dough procedure by adding 40 ppm FSMA and 30 ppm ascorbic acid to all doughs. Each lipolytic enzyme was dosed at the dosage know from previous trials to be the optimal dosage in the straight dough assay. The dosages were in the range from 0.17 to 0.5 mg enzyme protein per kg flour. The doughs were leavened for 45 minutes at 32° C., 86% relative humidity.

### Extraction

Just before the baking stage, the dough was transferred to the freezer (−18° C.). The samples were freeze dried and ground. A 4 g sample was extracted for 24 hours with 20 ml of an extraction medium prepared from 2500 ml 1-butanol and 100 ml 80 mM HCl, followed by centrifugation, filtration and evaporation of solvent. The residue was redissolved to a concentration of 10 mg/ml in MeOH/CHCl₃ (50:50) and analyzed by HPLC. The eluent consisted of chloroform (60-80%), methanol (19.5-34%), NH₄OH (0.5%) and water (0-6.0%), and the column was Microsorb-MV 100 Å-5 μm Si. Peak areas corresponding to MGDG and DGDG were determined.

### Correlation with Baking Performance

The lipolytic enzymes were tested in baking. Based on an evaluation of stability, loaf volume, crumb structure and dough properties, four of the ten lipolytic enzymes were found to show a relatively high degree of baking performance, whereas the other six lipolytic enzymes showed a poor baking performance. HPLC results for these two groups of lipolytic enzymes were found as follows:

| Number of lipolytic | Baking | MGDG | DGDG |
|---|---|---|---|
| enzymes | performance | degradation | degradation |
| 4 | Good | 100% | 10-28% |
| 6 | Poor | 48-92% | 0-74% |

The results indicate that the ability of a lipolytic enzyme to fully degrade MGDG can be used to predict its baking performance. DGDG degradation did not correlate well with baking performance.

1-6. (canceled)

7. A method of selecting a lipolytic enzyme for use as an additive to dough, comprising:
a) contacting at least one lipolytic enzyme
i) with a first lipid which is monogalactosyl diglyceride (MGDG), N-acyl phosphatidyl ethanolamine comprising an unsaturated acyl (APE), phosphatidyl ethanolamine comprising an unsaturated acyl (PE).
ii) and with a second lipid which is digalactosyl diglyceride (DGDG), phosphatidyl choline (PC), phosphatidyl myoinositol (PI), phosphatidyl serine (PS), phosphatidyl glycerol (PG), phosphatidyl ethanolamine not comprising an unsaturated acyl, N-acyl phosphatidyl ethanolamine not comprising an unsaturated acyl, N-acyl lysophosphatidyl ethanolamine (ALPE) or a triglyceride,
b) detecting hydrolytic activity of the enzyme towards ester bonds in the first and the second lipid,
c) comparing the activity towards the first lipid and the second lipid, and
d) selecting a lipolytic enzyme having a higher hydrolytic activity towards the first lipid than the second lipid,
with the proviso that the first lipid is not APE when the second lipid is PC.

8. The method of claim 7 wherein the first lipid comprises an unsaturated acyl, particularly polyunsaturated, which is preferably straight-chain with 16-20 carbon atoms, such as oleoyl, linoleoyl or linolenoyl.

9. The method of claim 7 wherein the second lipid comprises a saturated straight-chain acyl with 16-20 carbon atoms.

10. A method of preparing a dough, comprising:
a. selecting a lipolytic enzyme by the method of claim 7, and
b. adding the selected lipolytic enzyme to the dough.

11. A method of preparing a baked product, comprising:
c. preparing a dough by the method of claim 7, and
e) baking the dough.
